(19) United States
(12) Patent Application Publication
(10) Pub. No.: US 2026/0170639 A1
(43) Pub. Date: Jun. 18, 2026

(54) METHODS AND SYSTEMS FOR PROCESSING A DIGITIAL MEDICAL IMAGE TO PERFORM A DIAGNOSTIC MEASUREMENT

(71) Applicant: Mars, Incorporated, McLean, VA (US)

(72) Inventors: Fernando Rodrigues JUNIOR, São José dos Campos (BR); Mark Justin PARKINSON, Long Valley, NJ (US)

(21) Appl. No.: 18/980,468

(22) Filed: Dec. 13, 2024

(51) Int. Cl. G06T 7/00; G06T 3/60; G06T 7/60; G06T 7/73; G06V 10/25; G06V 10/764; G06V 10/82; G16H 50/20

(52) U.S. Cl. CPC G06T 7/0012; G06T 3/60; G06T 7/60; G06T 7/73; G06V 10/25; G06V 10/764; G06V 10/82; G16H 50/20; G06T 2207/10116; G06T 2207/20081; G06T 2207/20084; G06T 2207/30008; G06T 2207/30168; G06V 2201/033; G06V 2201/034

(57) ABSTRACT

A method for processing a digital medical image to perform a diagnostic measurement is described. The method may include receiving a digital medical image associated with a patient; detecting a first region of interest in the digital medical image, the first region including a first landmark and a second landmark; determining a first score of the digital medical image based on detected centers of the first and second landmarks, the first score representing a degree of laxity between the first landmark and the second landmark; determining a second score of the digital medical image, the second score representing a degree of proper positioning of the patient; determining a composite score based on the first score and the second score; and determining, based on the comparing of the composite score to the image selection threshold, the digital medical image is an optimal medical image for diagnosis.

### TECHNICAL FIELD

Various embodiments of this disclosure relate generally to systems and methods for processing a digital medical image to perform a diagnostic measurement and, more specifically, to systems and methods implementing one or more machine learning techniques to determine an optimal digital medical image for accurate diagnosis.

### BACKGROUND

Orthopedic conditions may affect the health of dogs for various breeds and sizes. For example, hip dysplasia may be a widespread and debilitating condition for dogs. Hip dysplasia may be characterized by an abnormal development of the hip joint, resulting in joint laxity, cartilage damage, and subsequent degenerative changes. Hip dysplasia may cause significant pain, lameness, and reduced mobility in affected dogs, severely impacting their quality of life. Recognizing and accurately diagnosing hip dysplasia may be crucial for identifying an appropriate treatment plan for dogs.

Conventional techniques for characterizing hip dysplasia may involve analyzing digital medical images of a hip of the dog to assign a diagnostic value. These digital medical images may be of varying quality and lower quality images may lead to less accurate diagnostic measurements. Further, conventional techniques may implement less precise processes for determining diagnostic measurements. The present disclosure is directed at addressing this and other drawbacks with existing techniques for analyzing digital medical images to determine orthopedic conditions of dogs.

The background description provided herein is for the purpose of generally presenting the context of the disclosure. Unless otherwise indicated herein, the materials described in this section are not admitted to be prior art, or suggestions of the prior art, by inclusion in this section.

### SUMMARY OF THE DISCLOSURE

According to certain aspects of the disclosure, methods and systems are disclosed for predicting a diagnostic measurement.

In some aspects, the techniques described herein relate to a method for processing a digital medical image to perform a diagnostic measurement. The method may include: receiving a digital medical image associated with a patient, the digital medical image showing bones and tissues of the patient; processing the digital medical image; applying a first classifier to classify the digital medical image to an assigned view; detecting a first region of interest in the digital medical image, the first region including a first landmark and a second landmark; detecting, by applying a first machine learning system, a center of the first landmark and a center of the second landmark; determining a first score of the digital medical image based on the detected centers of the first and second landmarks, the first score representing a degree of laxity between the first landmark and the second landmark; determining a second score of the digital medical image, the second score representing a degree of proper positioning of the patient, determining the second score including: determining a set of landmarks on the digital medical image; connecting the set of landmarks based on a predetermined sequence to form a set of edges; and determining the second score based on the set of edges; determining a composite score based on the first score and the second score; comparing the composite score to an image selection threshold; determining, based on the comparing of the composite score to the image selection threshold, the digital medical image is an optimal medical image for diagnosis; and generating and transmitting, to a user computing device, computer-executable instructions configured to cause the user computing device to construct and display a user interface that presents the first score and/or at least a portion of the digital medical image.

In some aspects, the techniques described herein relate to a method, wherein the digital medical image may be a radiograph image.

In some aspects, the techniques described herein relate to a method, wherein processing the digital medical image may include: determining a relevant region of the digital medical image, wherein the relevant region identifies a hip and a surrounding area of the hip of the patient.

In some aspects, the techniques described herein relate to a method, wherein processing the digital medical image may include: determining, by applying a rotation classifier machine learning system, a rotation angle associated with the digital medical image; and rotating the digital medical image based on the rotation angle to properly orient the digital medical image.

In some aspects, the techniques described herein relate to a method, wherein processing the digital medical image may include: applying the first classifier to the digital medical image to confirm the digital medical image is of a relevant view of the patient, wherein the relevant view includes a compression view, a distraction view, or a hip-extended view of a hip region of the patient.

In some aspects, the techniques described herein relate to a method, wherein applying the first classifier to classify the digital medical image to an assigned view may further include: classifying the digital medical image as one of a compression view, a distraction view, or a hip-extended view.

In some aspects, the techniques described herein relate to a method, wherein detecting the first region of interest in the digital medical may further include: determining an area of the digital medical image that includes a femoral head, an acetabulum, and a surrounding region of the femoral head and the acetabulum, wherein the first landmark may be the femoral head and the second landmark may be the acetabulum.

In some aspects, the techniques described herein relate to a method, wherein the first machine learning system may be a convolutional neural network configured to perform image segmentation.

In some aspects, the techniques described herein relate to a method, wherein determining the first score may include: determining a distance D representing a distance between the center of the first landmark and the center of the second landmark; determining a radius R representing a radius of the first landmark; and dividing the distance D by the radius R.

In some aspects, the techniques described herein relate to a method, wherein the first score may be a distraction index.

In some aspects, the techniques described herein relate to a method, wherein the digital medical image may further show medical equipment applied to the patient for diagnosis, and wherein the second score may represent a degree of proper positioning of a femoral head of the patient relative to the medical equipment in the digital medical image for diagnosis.

In some aspects, the techniques described herein relate to a method, wherein the second score may be determined using a second machine learning system, the second machine learning system may be a convolutional pose machine configured to analyze the degree of proper positioning of the patient relative to medical equipment applied to the patient for diagnosis.

In some aspects, the techniques described herein relate to a method, wherein the second score may include: a landmark score representing a ratio of valid landmarks detected; a device score indicating presence of medical equipment in the digital medical image; an alignment score indicating a degree of alignment between one or more legs of the patient represented by a first portion of the set of the edges and medical equipment represented by a second portion of the set of the edges; and a femoral head score indicating whether the first landmark is positioned between a set of bars of the medical equipment, wherein the device score, the alignment score, and the femoral head score may be calculated when the assigned view to which the digital medical image is classified as a distraction view image.

In some aspects, the techniques described herein relate to a computer system for processing a digital medical image to perform a diagnostic measurement, the computer system may include: at least one memory storing instructions; and at least one processor configured to execute the instructions to perform operations including: receiving a digital medical image associated with a patient, the digital medical image showing bones and tissues of the patient; processing the digital medical image; applying a first classifier to classify the digital medical image to an assigned view; detecting a first region of interest in the digital medical image, the first region including a first landmark and a second landmark; detecting, by applying a first machine learning system, a center of the first landmark and a center of the second landmark; determining a first score of the digital medical image based on the detected centers of the first and second landmarks, the first score representing a degree of laxity between the first landmark and the second landmark; determining a second score of the digital medical image, the second score representing a degree of proper positioning of the patient, determining the second score including: determining a set of landmarks on the digital medical image; connecting the set of landmarks based on a predetermined sequence to form a set of edges; and determining the second score based on the set of edges; determining a composite score based on the first score and the second score; comparing the composite score to an image selection threshold; determining, based on the comparing of the composite score to the image selection threshold, the digital medical image is an optimal medical image for diagnosis; and generating and transmitting, to a user computing device, computer-executable instructions configured to cause the user computing device to construct and display a user interface that presents the first score and/or at least a portion of the digital medical image.

In some aspects, the techniques described herein relate to a system, wherein the digital medical image may be a radiograph image.

In some aspects, the techniques described herein relate to a system, wherein processing the digital medical image may include: determining a relevant region of the digital medical image, wherein the relevant region may identify a hip and a surrounding area of the hip of the patient.

In some aspects, the techniques described herein relate to a system, wherein processing the digital medical image may include: determining, by applying a rotation classifier machine learning system, a rotation angle associated with the digital medical image; and rotating the digital medical image based on the rotation angle to properly orient the digital medical image.

In some aspects, the techniques described herein relate to a system, wherein processing the digital medical image may include: applying the first classifier to the digital medical image to confirm the digital medical image is of a relevant view of the patient, wherein the relevant view may include a compression view, a distraction view, or a hip-extended view of a hip region of the patient.

In some aspects, the techniques described herein relate to a system, wherein applying the first classifier to classify the digital medical image to an assigned view may further include: classifying the digital medical image as one of a compression view, a distraction view, or a hip-extended view.

In some aspects, the techniques described herein relate to a non-transitory computer-readable medium storing instructions that, when executed by a processor, cause the processor to perform operations for processing a digital medical image to perform a diagnostic measurement, wherein the operations may include: receiving a digital medical image associated with a patient, the digital medical image showing bones and tissues of the patient; processing the digital medical image; applying a first classifier to classify the digital medical image to an assigned view; detecting a first region of interest in the digital medical image, the first region including a first landmark and a second landmark; detecting, by applying a first machine learning system, a center of the first landmark and a center of the second landmark; determining a first score of the digital medical image based on the detected centers of the first and second landmarks, the first score representing a degree of laxity between the first landmark and the second landmark; determining a second score of the digital medical image, the second score representing a degree of proper positioning of the patient, determining the second score including: determining a set of landmarks on the digital medical image; connecting the set of landmarks based on a predetermined sequence to form a set of edges; and determining the second score based on the set of edges; determining a composite score based on the first score and the second score; comparing the composite score to an image selection threshold; determining, based on the comparing of the composite score to the image selection threshold, the digital medical image is an optimal medical image for diagnosis; and generating and transmitting, to a user computing device, computer-executable instructions configured to cause the user computing device to construct and display a user interface that presents the first score and/or at least a portion of the digital medical image.

It is to be understood that both the foregoing general description and the following detailed description are exemplary and explanatory only and are not restrictive of the disclosed embodiments.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary system environment in which the techniques discussed in the present disclosure may be implemented.

FIG. 2 depicts an exemplary flowchart of a process for determining an optimal digital medical image for medical diagnosis, according to one or more embodiments.

FIG. 3 depicts an exemplary diagram of process for determining an optimal digital medical image for medical diagnosis, according to one or more embodiments.

FIG. 4 depicts views of an exemplary digital medical image with side signs utilized for pre-processing the digital medical images, according to one or more embodiments.

FIG. 5 depicts exemplary distraction and compression views of exemplary digital medical images, according to one or more embodiments.

FIG. 6 depicts an exemplary view of a digital medical image with one or more identified landmarks, according to one or more embodiments.

FIG. 7 depicts an exemplary view of a digital medical image with an identified cavitation, according to one or more embodiments.

FIG. 8 depicts exemplary views of a digital medical image during landmark identification and analysis, according to one or more embodiments.

FIG. 9 depicts an exemplary view of a digital medical image with identified landmarks, according to one or more embodiments.

FIG. 10 depicts an exemplary flowchart of a method for processing a digital medical image to perform a diagnostic measurement, according to one or more embodiments.

FIG. 11 depicts an example of a computing device that may execute the techniques described herein, according to one or more embodiments.

### DETAILED DESCRIPTION OF EMBODIMENTS

Various embodiments of this disclosure relate generally to systems and methods for processing a digital medical image to perform a diagnostic measurement and, more specifically, to systems and methods implementing one or more machine learning techniques to determine an optimal digital medical image for accurate diagnosis.

The systems and methods described herein may receive a digital medical image (e.g., of an x-ray) directed at the hip region of an animal (e.g., a dog). The system may perform initial processing of the received digital medical image and classify the digital medical image into a corresponding image type or view. Upon processing and classifying the digital medical image, the system may determine a distraction index associated with the hip region shown in the digital medical image. Further, the system may apply one or more computational algorithms to examine the animal's positioning in the digital medical image in order to determine whether the animal is properly positioned for the purpose of accurate diagnosis (e.g., hip dysplasia). The system may analyze additional data associated with the patient and/or the digital medical image in order to determine whether the digital medical image is optimal or well-suited for accurate diagnosis. If the system determines that the digital medical image is optimal or well-suited for accurate diagnosis, the system may output the distraction index determined based on that digital medical image to one or more users, and may further analyze the distraction index and/or the digital medial image to generate a diagnosis and potentially one or more treatment options.

Conventional techniques for diagnosing hip dysplasia in animals may involve analyzing digital medical images of a hip region of an animal to determine a diagnostic value or measurement indicative of hip dysplasia. The identification of relevant aspects of the digital medical image may be performed by a user (e.g., a veterinarian). An exemplary method for analyzing hip dysplasia includes a PennHIP method.

Applying the PennHIP method as a diagnostic tool in the veterinary field may offer valuable insights into hip joint laxity and the likelihood of developing hip dysplasia. However, for the PennHIP method to be effective, the hip region of the subject needs to be properly positioned when taking the radiograph (e.g., X-ray). Applying the PennHIP method to a poorly-positioned radiograph may make it difficult to form a reliable diagnosis. The systems and methods described herein may utilize advanced machine learning techniques to determine digital medical images that are optimal or best-suited for assessing the risk of hip dysplasia, thus improving the accuracy and efficiency of hip dysplasia diagnosis.

One of the main risk factors for hip dysplasia is hip laxity, the degree of looseness of the hip joint. PennHIP is a method of measuring hip laxity in dogs using a special device that applies a force to the hips while taking radiographs. The distraction index (DI) is a numerical value that represents the ratio of the distance between the center of the femoral head and the acetabulum to the radius of the femoral head. The DI is a quantitative measure to assess the risk of canine hip dysplasia (CHD) by evaluating the laxity of a hip joint. The DI may be calculated based on radiographs (e.g., x-rays) of the hip joint or region, particularly by focusing on the displacement of the femoral head from the acetabulum under applied force. A lower DI may indicate a tighter hip joint, while a higher DI may indicate a more lax hip joint. A DI of 0 may be the tightest possible reading and a DI of 1 may represent a fully luxated hip. Dogs with a DI of >0.4 may be considered at an increased risk of developing hip dysplasia.

To determine a DI, an X-ray of a dog's hip may be analyzed. X-rays may be taken from various views or angles. For example, a compression view, a distraction view, and a hip-extended view of a hip region may be taken, providing a set of images to be evaluated. Certain views of x-rays (e.g., distraction views) may enable more accurate DI measurement. However, selecting the best distraction view for each hip joint can be challenging. The quality of the radiographs may depend on several factors, such as the positioning of the dog, the amount of force applied by the distractor, and the accuracy of the DI measurement. For example, if multiple digital medical images are analyzed and corresponding DI's are determined for each image, the highest DI may not accurately reflect the DI of the animal. This may be because aspects of the image (e.g., the positioning of the dog in the image, issues with the image, etc.) may lead to a less accurate DI. Therefore, it may be important to use standardized techniques and validated tools to obtain consistent and accurate measurements (e.g., DIs) of hip laxity in dogs.

The systems and methods described herein may implement one or more machine learning techniques to automate the selection of optimal x-ray images for diagnosing canine hip dysplasia. Through the implementation of advanced machine learning techniques, the system may compressively analyze factors such as animal and device position, laxity level, and the presence of cavitation. This may lead to improved efficiency, consistency, and diagnosis accuracy in diagnosing canine hip dysplasia, thereby benefiting veterinary practitioners and enhancing patient care in the field.

The systems and methods may incorporate a comprehensive and robust multi-stage process, encompassing different components that may leverage one or more machine learning techniques. The method may incorporate data preprocessing, view classification, landmark detection, and scoring to enhance the accuracy, efficiency, and reliability of an automated selection process for digital medical images.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

In the present disclosure, the term “animal” may refer to any animal that includes a hip. In an example, the term animal may include a dog.

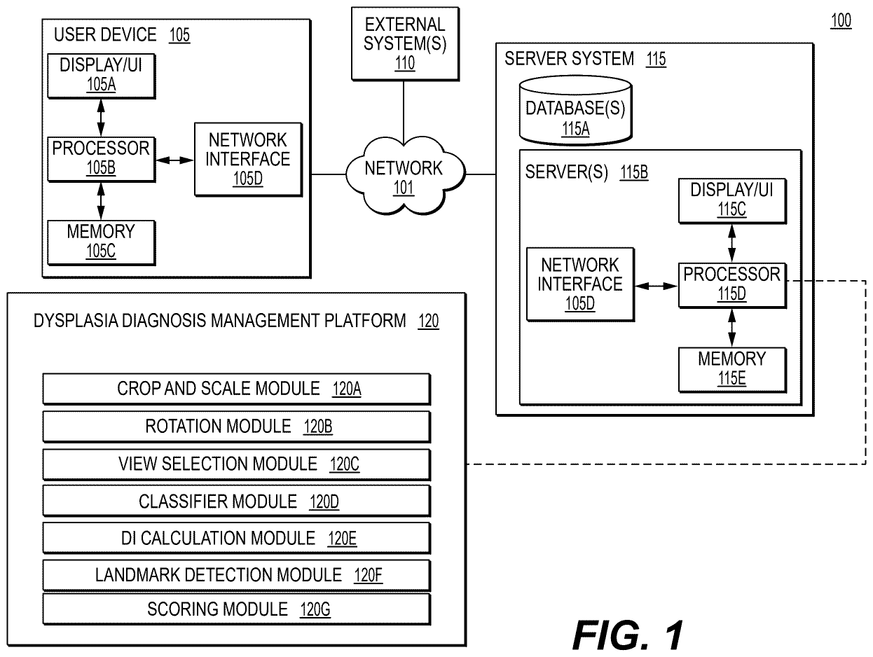

FIG. 1 depicts an exemplary system environment in which the techniques discussed in the present disclosure may be implemented. A user device 105, one or more external system(s) 110, and a server system 115 may communicate across a network 101. As will be discussed in further detail below, the server system 115 may communicate with one or more of the other components of the environment 100 across the network 101. The user device 105 may be associated with a user, e.g., a system manager, a pet owner, a veterinarian, a researcher, or the like. Although depicted as separate components in FIG. 1, it should be understood that a component or portion of a component in the environment 100 may, in some aspects, be integrated with or incorporated into one or more other components. For example, a portion of the display/UI 115C may be integrated into the user device 105 or the like. In some aspects, operations or aspects of one or more of the components listed above may be distributed amongst one or more other components. Any suitable arrangement and/or integration of the various systems and devices of the environment 100 may be used. Furthermore, it should be understood that while only one user device is shown in FIG. 1, the environment 100 may include a plurality of user devices 105 that are configured to communicate with the server system 115 over the network 101.

In some aspects, the components of the environment 100 may be associated with a common entity (e.g., a single business or organization, etc.). Alternatively, one or more of the components may be associated with a different entity than another. The systems and devices of the environment 100 may communicate in any arrangement. For example, the user device 105 may be associated with one or more clients or service subscribers, and the server system 115 may be associated with a service provider responsible for receiving and processing raw datasets from the one or more clients or service subscribers. As will be discussed herein, systems and/or devices of the environment 100 may communicate in order to collect digital medical images from a source (e.g., scanner, veterinary medical records, hospital systems, radiograph machines, etc.) and analyze the digital medical image to determine a diagnostic measurement.

The user device 105 may be configured to enable the user to access and/or interact with other systems in the environment 100. For example, the user device 105 may be a computer system such as, for example, a desktop computer, a laptop, a mobile device, a tablet device, a wearable device, etc. The user device 105 may include a display/user interface (UI) 105A, a processor 105B, a memory 105C, and/or a network interface 105D. The user device 105 may execute, by the processor 105B, an operating system (O/S) and at least one electronic application (each stored in memory 105C). The electronic application may be a desktop program, a browser program, a web client, or a mobile application program (which may also be a browser program in a mobile O/S), system control software, system monitoring software, software development tools, or the like. In some aspects, the electronic application(s) may be associated with one or more of the other components in the environment 100, such as the server system 115. The application may manage the memory 105C, such as a database, to transmit streaming data to the network 101. The display/UI 105A may be a touch screen or a display with other input systems (e.g., mouse, keyboard, etc.) so that the user(s) may interact with the application and/or the O/S. The network interface 105D may be a TCP/IP network interface for, e.g., Ethernet or wireless communications with the network 101. The processor 105B, while executing the application, may generate data and/or receive user inputs from the display/UI 105A and/or receive/transmit messages to the server system 115, and may further perform one or more operations prior to providing an output to the network 101.

The electronic application, executed by the processor 105B of the user device 105, may generate one or more points of data that can be accessed, viewed, and/or interacted with by a user of the user device 105. More particularly, the electronic application may be associated or in communication with a dysplasia diagnosis management platform 120 that is hosted, managed and/or supported by one or more of the server system 115. A user of user device 105 may interact with dysplasia diagnosis management platform 120 via the electronic application to obtain a diagnostic measurement (e.g., a DI), processed or annotated digital medical images, a diagnosis, and the like, which may be displayed on a user interface in an intuitive and easily-navigable manner. In some aspects, the dysplasia diagnosis management platform 120 may leverage one or more trained Artificial Intelligence Processing Modules (AIPMs) to process digital medical images, classify images, determine diagnostic measurements, evaluate and score digital medical images, and/or determine diagnoses and treatment plans, as further described herein. The dysplasia diagnosis management platform 120 may include a set of modules configured to analyze a received digital medical image, process the image, determine a DI, and determine an overall score for the digital medical image as will be described in greater detail below. These diagnostic measurements and overall score may then be provided to the user device 105 via the electronic application.

The external system(s) 110 may be, for example, one or more third party and/or auxiliary systems that integrate and/or communicate with the server system 115 in performing various information extraction tasks. The external system(s) 110 may be in communication with other device(s) or system(s) in the environment 100 over the network 101. For example, the external system(s) 110 may communicate with the server system 115 via API (application programming interface) access over the network 101, and also communicate with the user device 105 via web browser access over the network 101. Non-limiting examples of the external systems 110 may include one or more x-ray machines, scanners, and/or databases containing digital medical images of animals.

In various aspects, the network 101 may be a wide area network (“WAN”), a local area network (“LAN”), a personal area network (“PAN”), or the like. In some aspects, the network 101 includes the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

In some aspects, the server system 115 includes and/or interacts with an application programming interface for exchanging data to other systems, e.g., one or more of the other components of the environment. The server system 115 may include and/or act as the host for an application platform (e.g., dysplasia diagnosis management platform 120, etc.) that may be accessible by the user device 105.

The server system 115 may include one or more database(s) 115A and one or more server(s) 115B. The server system 115 may be a computer, system of computers (e.g., rack server(s)), and/or or a cloud service computer system. The server system 115 may store or have access to database(s) 115A (e.g., hosted on a third party server or in memory 115E). The server(s) 115B may include a display/UI 115C, a processor 115D, a memory 115E, and/or a network interface. The display/UI 115C may be a touch screen or a display with other input systems (e.g., mouse, keyboard, etc.) for an operator of the server(s) 115B to control the functions of the server(s) 115B. The server system 115 may execute, by the processor 115D, an operating system (O/S) and at least one instance of a servlet program (each stored in the memory 115E). When the user device 105 transmits input to the server system 115 (e.g., pet owner inputs, etc.), the received dataset and/or dataset information may be stored in the memory 115E or the database(s) 115A. The network interface may be a TCP/IP network interface for, e.g., Ethernet or wireless communications with the network 101.

The processor 115D may include and/or execute instructions to implement a dysplasia diagnosis management platform 120, which may include a crop and scale module 120A, a rotation module 120B, a view selection module 120C, a classifier module 120D, a DI calculation module 120E, a landmark detection module 120F, and/or a scoring module 120G. In some embodiments, one or more of the modules of the dysplasia diagnosis management platform 120 may be located or stored on separate or external servers that may be accessed by the server system 115 through network 101. Alternatively, some or all of the foregoing modules may be submodules of other modules within each other or may be resident on other components of the environment 100.

The crop and scale module 120A may be configured to receive digital medical image(s) of a patient (e.g., an animal such as a dog). The digital medical images may be x-rays focusing on a specific region of the patient's body, the region including bones and tissues of an animal. The digital medical images may optimally include a hip area of the patient. The digital medical images may be obtained from an x-ray machine, from a server storing digital medical images, or may be uploaded by a user (e.g., through external systems 110 or by user device 105). The crop and scale module 120A may apply one or more algorithms to prepare the digital medical image for further analysis. The crop and scale module 120A may detect X-ray edges, remove any extra background, and scale an image to a correct size. Further, the crop and scale module 120A may remove padding in the digital medical image caused by a collimator. The crop and scale module 120A may incorporate a machine learning system such as a region-based convolution neural network (R-CNN). The R-CNN may localize the X-ray image and remove any unnecessary background or artifacts (e.g., cropping the image). The cropping may ensure that only relevant regions of interest (ROI) containing essential anatomical structures remain (e.g., a femoral head, acetabulum, and surrounding bones and tissues). This may reduce the amount of unnecessary information and potential inferences in later stages of processing and analyzing the digital medical image. The crop and scale module 120A may, upon performing cropping, scale the image. This may include applying an algorithm that resizes the cropped image to a standardized dimension. This may ensure consistency in the size and scale of the x-ray images, as smaller images may lack the necessary level of detail for accurate analysis.

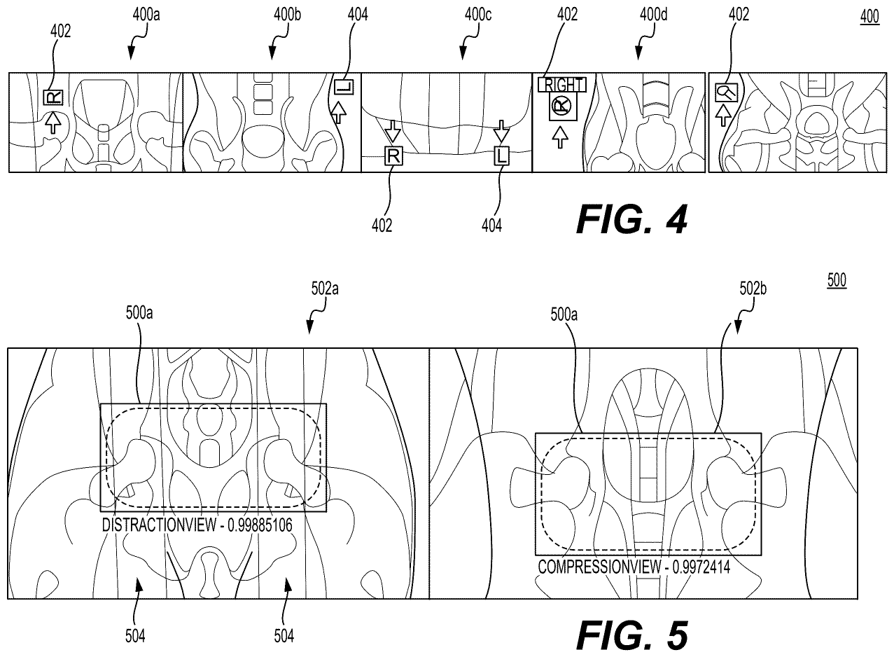

The crop and scale module 120A may then output the cropped and scaled images to the rotation module 120B. The rotation module 120B may include a rotation classifier machine that incorporates an R-CNN. The R-CNN may have been trained on a labeled dataset that includes a multitude of digital medical images (e.g., hundreds, thousands, or more) with annotations for a variety of left and right label images (as shown in FIG. 4). The rotation module 120B may be configured to rotate the digital medical image to a standardized alignment. In one embodiment, the rotation module 120B may be configured to rotate the digital medical image based on a rotation angle the module 120B determines. In one embodiment, the rotation module 120B may rotate the digital medical image based on specific markers within the x-ray images (e.g., right and left labels) that provide information about the orientation of the digital medical image. The rotation module 120B may orient the digital medical image, as a uniform orientation may be necessary to compare and analyze a plurality of digital medical images. Further, separate analysis may be applied on both the right side and the left side of a hip joint of the digital medical images. Therefore, it may be necessary to ensure that the correct side of the hip joint is analyzed at further processing steps. For example, the rotation module 120B may be configured to rotate the digital medical image in 90-degree increments. In other embodiments, each increment can be smaller or larger than 90 degrees. The rotation module 120B may further recognize that the image is in a correct orientation and not apply a rotation. The rotation module 120B may be configured to output digital medical images that are properly oriented (e.g., in a standard alignment).

The rotation module 120B may be configured to output digital medical images to a view selection module 120C. The view selection module 120C may include a deep learning-based classifier configured to distinguish digital medical images that are utilized for applying the PennHIP method from digital medical images of other body parts (e.g., digital medical images that are not suitable for the application of the PennHIP method). For example, the view selection module 120C may be configured to classify and determine which digital medical images are of a compression view, a distraction view, or a hip-extended view of a hip (referred to herein as PennHIP images). In an example, the classifier of the rotation module 120B may have been trained on a labeled dataset of a multitude of X-ray images (e.g., hundreds, thousands, or more), encompassing PennHIP images and non-PennHIP images. The classifier may leverage a Resnet 50 based model, wherein the classifier may be configured to recognize the unique features and patterns specific to PennHIP images. In an example, of the determined images to be PennHIP images, the images may be identified as either (1) a compression view or distraction view; a (2) a hip-extended view; or (3) an alternative view. Examples of the compression view and the distraction view are shown in FIG. 5. The determined view may be assigned to the received digital medical images.

The view selection module 120C may output the identified images (e.g., PennHIP images) to the classifier module 120D. The classifier module 120D may include a deep learning-based classifier separate from the classifier in the view selection module 120C. The classifier in the classifier module 120D may be configured to differentiate between compression view and distraction view within the categorized PennHIP digital medical images. The compression view and distraction view may exhibit similar visual characteristics and shared common features, making it difficult for a single classifier to accurately differentiate between them and other limbs in the same vector space. The classifier in the classifier module 120D may include a second layer making it possible to attend to the region of interest (ROI) encompassing the femoral head and acetabulum, as these structures are crucial for extracting the distraction index and distinguishing between the views. The classifier module 120D may enhance the system's capability to distinguish between different types of images at a more granular level, enabling it to distinguish between the compression and distraction views more efficiently, compared to when only the view selection module 120C is used. In some examples, the classifier module 120D may only be applied when a received image is determined to be in compression view or distraction view from the view selection module 120C. Upon application of the view selection module 120C and the classifier module 120D, a digital medical image may be assigned a view of either a compression view, a distraction view, a hip-extended view, or an alternative view. The alternative view images may not be further analyzed. The compression view, a distraction view, and a hip-extended view images may be considered approved images for further analysis, including diagnostic measurement.

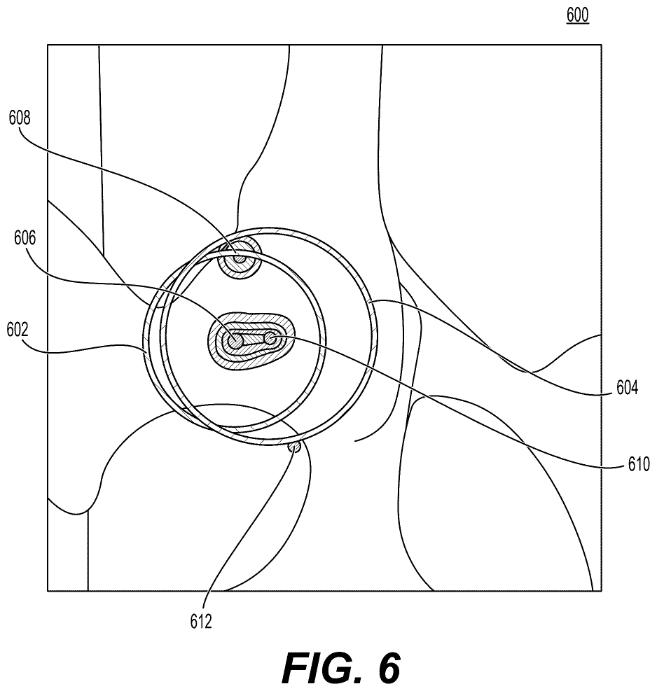

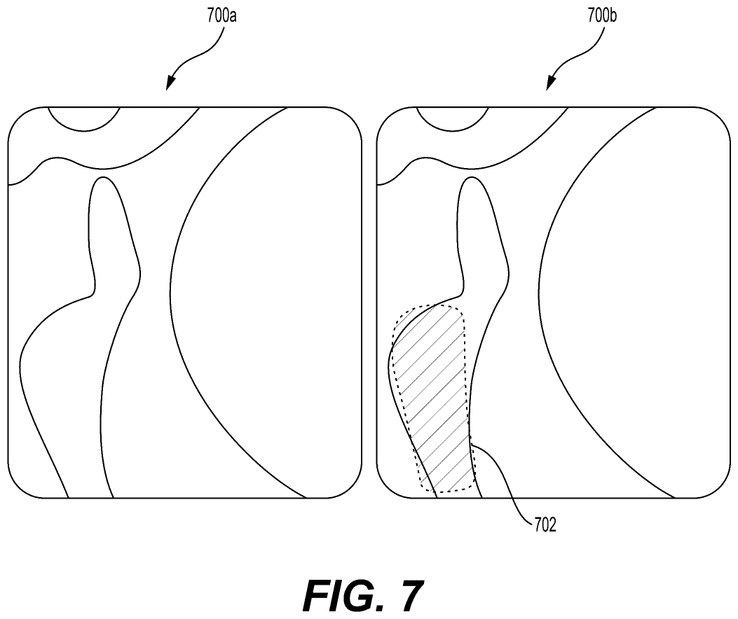

The view selection module 120C and the classifier module 120D may output the approved images to the DI calculation module 120E. The DI calculation module 120E may be configured to determine a DI for the received digital medical image. In order to calculate the DI accurately, it may be necessary to localize the acetabulum and femoral head in the digital medical image. The DI calculation module may include a first machine learning system, which may be a deep regression model. The first machine learning system may be utilized to extract one or more landmarks from the digital medical image. For example, the first machine learning algorithm may be configured to identify a first landmark (e.g., a femoral head) and a second landmark (e.g., an acetabulum) from the digital medical image. The first machine learning algorithm may determine a center and radius of the first and second landmarks respectively. The first machine learning system may incorporate a CNN such as a U-Net architecture that combines a deep encoder-decoder structure. This design may integrate global landmark configurations with local high-resolution feature response, allowing for precise landmark extraction. The first machine learning system may use a multi-channel heatmap for landmark detection, capturing the essential locations and characteristics. The deep encoder-decoder structure may analyze both global and local features, enabling a comprehensive understanding of landmark arrangement. By leveraging these features, the model may accurately determine the center and radius of the landmarks. An exemplary output of the first machine learning system is shown in FIG. 6. The DI calculation module 120E may further be configured to determine a first score of the digital medical images (e.g., a DI) based on the outputs of the first machine learning system. For example, the DI calculation module 120E may be configured to compute a DI by calculating a distance d representative of the distance between the center of the femoral head and the center of the acetabulum in the distraction view, determining a radius r of the femoral head, and dividing d by r.

The dysplasia diagnosis management platform 120 may further include a module to determine any cavitation in a digital medical image. The cavitation may refer to the formation of a gas-filled void within a joint. In some rare cases, cavitation may occur during the PennHIP distraction procedure when the distractor device applies lateral force to the hips, creating negative pressure in the synovial fluid (e.g., the cavitation may occur during the capturing of the digital medical image). This may lead to the formation of an air bubble, which may be visible in the radiograph (as shown in FIG. 7). Such cavitation may be detected by the system as it may inflate a DI measurement, making the DI less reliable. For example, a cavitated joint may show a false increase in laxity, leading to an inaccurate evaluation of the risk for hip dysplasia. Detection and accounting for cavitation may ensure that the DI measurement remains valid and reflects the true condition of the hip joint. Table 1 below shows an exemplary rate of cavitation occurrence in PennHIP evaluation.

TABLE 1

| Hip | N | % | 95% CI |
|---|---|---|---|
| Either | 279 | 4.2 | 3.7-4.7 |
| Right | 145 | 2.2 | 1.8-2.6 |
| Left | 156 | 2.3 | 2.0-2.7 |
| Both | 18 | .27 | 0.17-0.44 |

The dysplasia diagnosis management platform 120 may further include a landmark detection module 120F. The landmark detection module 120F may be configured to analyze the positioning of the landmarks identified within the digital medical image. For example, a proper position of the animal may be critical to ensure accurate and reliable measurements and/or diagnosis. The landmark detection module 120F may analyze the positioning of the animal, or its targeted anatomical structure(s), within a digital medical image. The landmark detection module 120F may incorporate a deep learning algorithm to identify a set of key anatomical landmarks (e.g., sixteen anatomical landmarks). These landmarks may allow for the evaluation of leg parallelism, the positioning of the femoral head relative to or within the distractor bars, and other critical alignment criteria. The landmark detection module may include a Convolutional Pose Machine (CPM), which may be configured to identify points by regressing Gaussian heatmaps, representing the probability distribution of each landmark's location. The CPM may iteratively refine the landmark positions through a series of convolutional layers, improving accuracy by using both local image features and contextual information from the surrounding area. The identified points (e.g., landmarks) may be grouped into joints, making it possible to distinguish between left and right femoral head, distraction bars, and other structures. Using these groupings, the CPM may apply a heuristic approach to measure the angles and compare the alignment between the right and left legs in distraction views. The landmark detection module 120F may determine one or more scores representative of the degree of proper positioning of the animal in the digital medical image. This process may ensure that the hip positioning meets the necessary criteria for accurate DI calculation.

The dysplasia diagnosis management platform 120 may further include a scoring module 120G. The scoring module 120G may be configured to score a digital medical image and assign a weighted value to a digital medical image that incorporates a DI and various aspects of a digital medical image (e.g., it may incorporate factors such as detection of cavitation, identification of anatomical landmarks, alignment of legs with the distractor bars, and/or the assigned perspective of the image). To arrive at this value, various aspects of the digital medical image may be evaluated and scored, and the value may be calculated based on a weighted equation taking the multiple scores into account. For example, the scoring module 120G may obtain scores from other modules in the dysplasia diagnosis management platform 120 (e.g., the DI may be obtained from the DI calculation module 120E). The scoring module 120G may be configured to apply an algorithm to identify the overall score of each digital medical image as will be described in greater detail below. For example, the scoring module 120G may be utilized to score a set of digital medical images and to determine an optimal image to determine a DI for a dog. In another example, the scoring module 120 G may be utilized to determine whether the weighted score for a digital medical image is greater than a threshold value and whether the digital medical image is considered an adequate image for determining a DI for a dog based on the comparison of the weighted score to the threshold value.

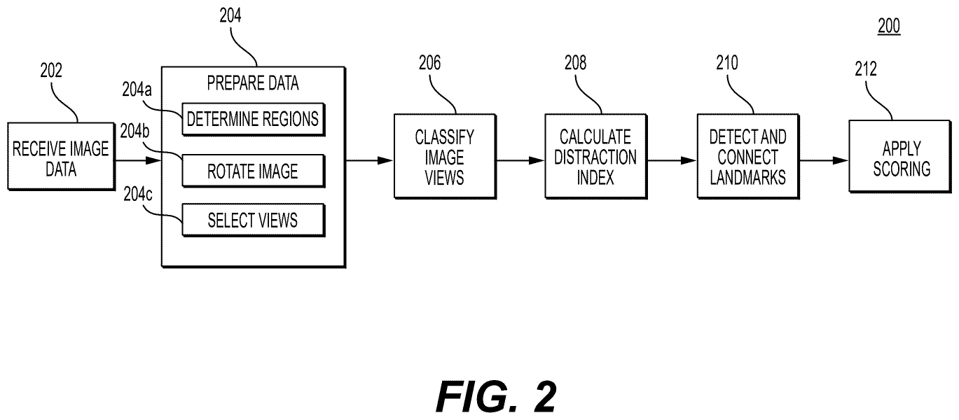

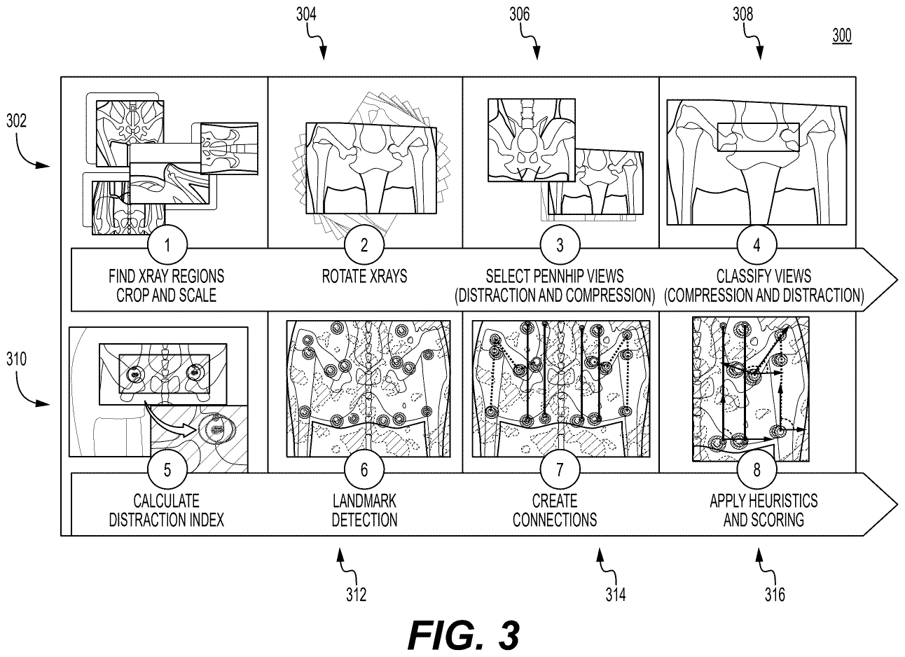

FIG. 2 depicts an exemplary flowchart of a process for determining an optimal digital medical image for medical diagnosis, according to one or more embodiments. The process described in FIG. 2 may be implemented by the environment 100 of FIG. 1. FIG. 3 is an exemplary diagram 300 of process described in FIG. 2, illustrating how the digital medical images may be processed by the corresponding modules and/or algorithms. The diagram 300 of FIG. 3 is referenced to illustrate the steps of the flowchart 200 of FIG. 2. Further, FIG. 4 through FIG. 9 depict various digital medical images illustrating aspects of the processes depicted in flowchart 200.

At step 202, the dysplasia diagnosis management platform 120 may receive a digital medical image. The digital medical image may capture a specific region (e.g., anatomical structure(s)) within the patient's body, the region including bones and tissues of a patient. The patient may for example be an animal such as a dog. In some examples, the digital medical image may be a radiograph image such as an X-ray. In some examples, the digital medical image may be of the hip region of an animal. The digital medical image may further show medical equipment applied to the patient for diagnosis.

In an example, the digital medical image of step 202 may be captured by placing a dog under sedation or anesthesia to minimize muscle tension and prevent voluntary movement. The dog may then be radiographed in one or more positions (e.g., corresponding to a distraction view, a compression view, and a hip extension view). In some examples, a distractor may be applied to the dog's body prior to taking the x-ray images. In these cases (e.g., distraction view images), one or more fixed bars (e.g., the distractor's lateral band) may be depicted in the image (as shown in FIG. 5). In some examples, the distraction view digital medical images may provide a more optimal image for determining an accurate DI.

At step 204, the dysplasia diagnosis management platform 120 may process, or prepare, the digital medical image. Such processing or preparation may include one or more of step 204a, step 204b, and step 204c. Step 204a may be implemented by the crop and scale module 120A, step 204b may be implemented by the rotation module 120B, and step 204c may be implemented by the view selection module 120C of FIG. 1. At step 204a, the crop and scale module 120A may determine a relevant region of the digital medical image, wherein the relevant region identifies a hip and a surrounding area of the hip (collectively, a hip region) of the patient. At step 204a, the crop and scale module 120A may also apply a scaling algorithm to standardize the size of the digital medical image. Furthermore, the crop and scale module 120A may apply an algorithm to crop the image to include only the relevant regions of the digital medical image. At step 204b, the rotation module 120B may determine, by applying a rotation classifier machine learning system, a rotation angle associated with the digital medical image, and rotate the digital medical image based on the rotation angle to properly orient the digital medical image. The digital medical image may be rotated in increments (e.g., 10-degree increments, 20-degree increments, 30-degree increments, 40-degree increments, 60-degree increments, 90-degree increments, etc.). For example, FIG. 4 depicts views 400 of exemplary digital medical images with a side sign. These digital medical images may be the images processed at step 204b to be properly oriented. For example, digital medical images 400a, 400b, 400c, and 400d are exemplary digital medical images that may need to be rotated to a proper orientation. In one embodiment, the rotation classifier machine learning system may recognize the letters (e.g., letter R 402 and letter L 404) in the respective digital medical images and determine the initial orientation of the respective digital medical images based on the recognized letters. Based on this determined initial orientation, a rotation angle may be determined and applied to the images to properly orient the images.

At step 204c, the view selection module 120C may apply a classifier to the digital medical image to confirm whether the digital medical image is of a relevant view of the patient, wherein a relevant view corresponds to a compression view, a distraction view, or a hip-extended view of the hip region of the patient. FIG. 3 displays aspects of steps 204a and 204b as applied to exemplary images. For example, images 302 and 304 of diagram 300 are exemplary images that have been processed through steps 204a and 204b, respectively.

At step 206 the classifier module 120D may classify the image views. The classifier module 120D may apply a first classifier to classify the digital medical image to an assigned view. This may include classifying the digital medical image as one of a compression view, distraction view, or hip-extended view of the hip region of the patient. The classifier module 120D may further apply a second classifier to distinguish medical images of a compression view from those of a distraction view.

The method of FIG. 2 may further include the step of detecting a first region of interest in the digital medical image, the first region including at least a first landmark and a second landmark. For example, the region of interest detection step may be performed once the digital medical images are classified (e.g., step 206). The region detected at this step may cover a smaller, more specific region (which is more effective or targeted for the purpose of determining a DI or the risk of hip dysplasia) than the relevant region that may have been identified during the image processing stage (e.g., step 204). This region of interest detection step may include determining an area of the digital medical image that includes a femoral head, an acetabulum, and a surrounding region of the femoral head and acetabulum, wherein the first landmark is the femoral head and the second landmark is the acetabulum. The first region of interest may for example be determined by a machine learning system, such as a classifier. The first region of interest may be identified by a bounding box marking the first region of interest in the digital medical image. Image 308 of FIG. 3 illustrates an exemplary bounding box depicting a first region of interest including at least a first landmark and a second landmark.

FIG. 5 depicts distraction and compression views 500 of exemplary digital medical images that may be processed. The digital medical image 502a may be of the distraction view and the digital medical image 502b may be of a compression view. As discussed above with reference to step 206, bounding boxes 500a may define the relevant region (e.g., determined at step 206) for the respective medical images. In particular, the digital medical image 502a may include a fixed bar 504 that may have been applied on the patient at the time the digital medical image was captured. The methods described herein may detect the position of the fixed bar 504 and use the detected position in analyzing the positioning and alignment of the patient (or the hip region of the patient relative to the bar 504) as discussed below. FIG. 3 displays aspects of step 206 as applied to exemplary images. For example, images 306 of diagram 300 displays particular images being assigned to a particular image view (e.g., one of a compression view, distraction view, or hip-extended view of the hip region of the patient). Image 308 is an exemplary image that has been further classified as either a compression view or a distraction view, with a bounding box identifying a portion of the image (e.g., a region of interest) that is most pertinent to the determination of DI or the risk of hip dysplasia.

At step 208, the DI calculation module 120E may determine a first score of the digital medical image. The first score may be a DI and may be calculated based on outputs from a first machine learning system. The first machine learning system may implement a deep regression model which may employ a U-NET architecture. Step 208 may further include detecting, by applying the first machine learning system, a center of the first landmark and a center of the second landmark. Step 208 may additionally include determining the first score of the digital medical image based on the detected centers of the first and second landmarks, the first score representing a degree of laxity between the first landmark and the second landmark. Determining the first score, which may be a DI as discussed above, may include the following steps: determining a distance d, the distance d representing a distance between the center of the first landmark and the center of a second landmark, determining a radius r representing a radius of the first landmark and, dividing the distance d by the radius r. The equation utilized to determine the DI may be represented as:

$$DI = \frac{d}{r}$$

where d may represent the distance between the center of the femoral head and the center of the acetabulum in the distraction view (i.e., the displacement under applied force), and r may represent the radius of the femoral head.

The DI may be a dimensionless value that ranges from 0 to 1. A DI of zero may indicate no laxity, with the femoral head tightly seated in the acetabulum. A DI of 1 may indicate complete luxation, where the femoral head is fully displaced from the acetabulum. Higher DI values may correlate with increased joint laxity and a higher risk of developing hip dysplasia and osteoarthritis. Specifically a DI greater than 0.5 may be associated with a higher probability of hip dysplasia, while a DI below 0.3 may suggest a low risk. The step 208 may be applied twice for a single digital medical image, once for a first portion of the hip region in the digital medical image and once for a second portion of the hip region in the digital medical image. For example, a DI_{left} and a DI_{right} may be determined for each digital medical image.

FIG. 6 depicts an exemplary view of a digital medical image 600 with one or more identified landmarks. FIG. 6 may depict an exemplary output of the first machine learning system from step 208. The outputted digital medical image 600 may include an outlined femoral head 602 and an outlined acetabulum 604. The machine learning system may have identified a center point 606 of the femoral head and an outer circumference 608 relative to the center point 606. The machine learning system may have further identified a center point 610 of the acetabulum and the circumference 612 relative to the center point 610. This output from the first machine learning system may then be utilized to determine a DI. FIG. 3 displays aspects of step 208 as applied to an exemplary image. For example, images 310 of diagram 300 are exemplary images being processed through the landmark detection of step 208 as described above.

The method illustrated in the flowchart 200 may further include (between steps 208 and 210 or between steps 208 and 212), a process of applying a cavitation detection algorithm to identify one or more cavitated joints in the digital medical image. One or more machine learning techniques may be implemented to identify the cavitated joint(s) in the digital medical image. FIG. 7 depicts an exemplary view of a digital medical image 700a prior to applying the cavitation detection algorithm. The cavitation detection algorithm may be applied to the digital medical image to create digital medical image 700b, which identifies a cavitation 702. The digital medical image may include or be associated with metadata indicating the presence and/or the location of the cavitation. In one embodiment, a digital medical image may be assigned a cavitation score C, where the image is assigned a value of 1 if no cavitation was identified and a score of 0 if one or more cavitations were identified. Further, a cavitation score may be determined separately for a first portion of the hip region and a second portion of the hip region of a digital medical image. For example, the method may include determining a C_{left} and a C_{right} for each digital medical image.

At step 210, the landmark detection module 120F may detect and connect a plurality of landmarks in the digital medical image. Step 210 may include determining one or more scores (e.g., a second score) that represent a degree of proper positioning of the patient or the hip region of the patient within the digital medical image, based on the landmarks detected and connected in the digital medical image. The second score may represent a degree of proper positioning of the patient's femoral head relative to the medical equipment in the digital medical image for diagnosis. The second score may be determined by a second machine learning system, the second machine learning system being a CPM, configured to analyze the degree of proper positioning of the patient relative to the medical equipment applied to the patient for diagnosis. The second score may be generated for each of a first portion of the hip region and a second portion of the hip region (e.g., the left side and the right side). In one embodiment, step 210 may include determining a set of landmarks on the digital medical image, connecting the set of landmarks based on a predetermined sequence to form a set of edges, and determining the second score based on the set of edges. The second score may represent the alignment of the legs relative to medical equipment (e.g., distractor bars used in the PennHIP evaluation) within the digital medical image. The second score may be reflect the evaluation of leg parallelism, the positioning of the femoral head relative to or within the distractor bars, and other alignment criteria.

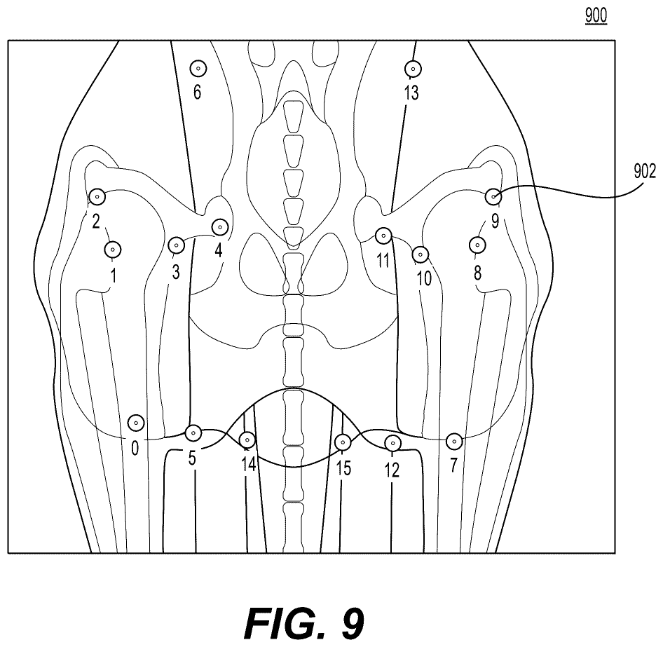

Based on the determined set of landmarks, a ratio R_{landmarks} may be identified at step 210. R_{landmarks} may represent the number of landmarks identified (as described in FIG. 9 below) divided by the number of potential landmarks to be identified (e.g., 16 landmarks). If all landmarks are identified, a R_{landmarks} may be 1.

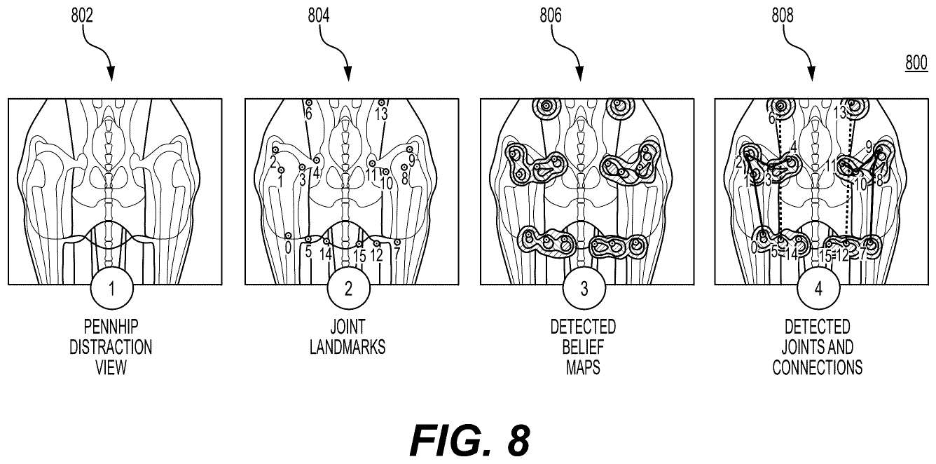

Step 210 is further illustrated by FIG. 8, which depicts exemplary views 800 of a digital medical image being processed through the landmark identification and analysis of step 210. View 802 shows an exemplary medical image that may be analyzed. View 804 depicts the identified landmarks, as will be described in greater detail with reference to FIG. 9. View 806 depicts the detected belief maps of the identified joint landmark locations. The identified joint landmark locations may be identified by the second machine learning system. A belief map may be a heatmap that identifies a confidence level of a landmark being located at a certain location in the image. View 808 may depict the landmarks that are connected to form edges, which may be further analyzed by the second machine learning system. In FIG. 3 illustrates aspects of step 210. For example, image 312 depicts the detected landmarks and image 314 depicts the landmarks connected to form edges for further analysis.

FIG. 9 depicts an exemplary view of a digital medical image 900 with landmarks 902 identified by the second machine learning system of step 210. The second machine learning system may be configured to identify 16 key anatomical landmarks 902 as represented in table 2 below:

TABLE 2
Landmarks and Descriptions

FEMUR LEFT BOTTOM: 0
FEMUR LEFT TOP: 1
FEMURCONNECTION2 LEFT: 2
FEMURCONNECTION1 LEFT: 3
FEMURHEAD LEFT: 4
DEVICEBAR LEFT BOTTOM LEFT: 5
DEVICEBAR LEFT TOP LEFT: 6
FEMUR RIGHT BOTTOM: 7
FEMUR RIGHT TOP: 8
FEMURCONNECTION2 RIGHT: 9
FEMURCONNECTION1 RIGHT: 10
FEMURHEAD RIGHT: 11
DEVICEBAR RIGHT BOTTOM RIGHT: 12
DEVICEBAR RIGHT TOP RIGHT: 13
DEVICEBAR LEFT BOTTOM RIGHT: 14
DEVICEBAR RIGHT BOTTOM LEFT: 15

In one embodiment, the second score identified at step 210 may be made up of a landmark score (R_{landmarks}), a device score (B_{right}, B_{left}), an alignment score (A_{left}, A_{right}), and a femoral head score (C_{right}, C_{left}). Each of these scores may be determined by the second machine learning system. More particularly, each of these scores may be determined by a respective scoring algorithm or model of the second machine learning system. As discussed above, the landmark score (R_{landmarks}) may represent the ratio of valid landmarks detected out of the potential landmarks. For example, if 12 landmarks out of potential 16 landmarks were identified, a score of 0.75 may be assigned. In some embodiments, the device score, alignment score, and femoral head score may be calculated only when the digital medical image is a distraction view image. In other embodiments, the device score, alignment score, and femoral head score may be calculated when the digital medical image is a compression view, a distraction view, or a hip-extended view of a hip region of the patient. The device score (B_{right}, B_{left}) may represent whether a set of distractor bars were detected in the digital medical image, wherein a score of 1 is assigned if the bars are detected and a score of 0 is detected if no bars are identified. The alignment score (A_{left}, A_{right}) may be determined by the second machine learning system based on the angles of the detected landmarks and connections. A perfect alignment may be assigned an alignment score of 1, where a score of 0 may represent a complete misalignment in the identified landmarks. The femoral head (C_{right}, C_{left}) score may represent whether the first landmark (e.g., the femoral head) was identified and located between the distractor bars within the digital medical image. If the first landmark is within the distractor bars, the femoral head score may be assigned a 1. If the first landmark is not in between the distractor bars, it may be assigned a score of 0.

Step 210 may be performed to measure the degree of proper positioning of the animal in the digital medical image, which may be critical to ensure accurate and reliable results. Correct positioning of the animal in the digital medical image may ensure accurate laxity measurements, diagnostic consistency, and predictive value for canine hip dysplasia.

Step 212 may include determining a composite score for the digital medical image. Step 212 may be implemented by the scoring module 120G. The composite score may include combining the individual scores generated by the different modules discussed above. The individual scores may be weighted in generating the composite score, based on the importance of each individual score in evaluating the selectivity of the digital medical image. In one embodiment, the composite score may further include the cavitation score. A composite score may be identified for each leg (e.g., each side, such as left or right) in the digital medical image.

The composite score may be based on a set of factors including, but not limited to, the detection of cavitation, the identification of anatomical landmarks, the alignment of legs relative to the distractor bars, DI, and the correct positioning of the femoral heads relative to or within the bars. A higher composite score may indicate a more optimal image, which may lead to a more accurate DI measurement.

The composite score for each leg may be determined based on the equations below.

$${S}_{left} = \frac{1}{9} ( {R}_{l a n d m a r k s} + {B}_{left} + {A}_{left} + {I}_{left} + 4 * {DI}_{left} + {C}_{left} )$$

$${S}_{r ight} = \frac{1}{9} ( {R}_{l a n d m a r k s} + {B}_{r ight} + {A}_{r ight} + {I}_{r ight} + 4 * {DI}_{r ight} + {C}_{r ight} )$$

where
S_{left} and S_{right} are the composite scores for the left and right legs, respectively;
R_{landmarks} is the ratio of valid landmarks detected;
B_{left}, B_{right} are the scores for detecting the device bars;
A_{left}, A_{right} are the alignment scores between the leg and the device bars;
I_{left}, I_{right} indicate whether the femoral head is within the bars;
DI_{left}, DI_{right} are the Distraction Index values for the left and right legs, respectively; and
C_{left}, C_{right} are the cavitation scores (e.g., 1 if no cavitation, 0 otherwise).

As shown above, the detection of anatomical landmarks may be evaluated based on a ratio, with 1 indicating that all landmarks have been identified. The position and alignment of the femoral head relative to the distractor bars may also considered, with higher scores given when they are positioned parallel to each other. Lastly, a score may be assigned based on whether the femoral heads are correctly positioned within the bars.

FIG. 3 illustrates aspects of step 212 as applied to exemplary images. For example, image 316 of diagram 300 illustrates a variety of factors analyzed by the second machine learning model to generate the respective individual scores and ultimately generate the composite score of step 212.

In some embodiments, the method of flowchart 200 may further include comparing the composite score to an image selection threshold. For example, the techniques of steps 202-212 may be applied to a set of digital medical images. Each of the scores determined at step 212 may be compared to the image selection threshold, in order to determine an optimal digital medical image. The method may further include determining, based on the comparing of the composite score to the image selection threshold, whether the digital medical image is an optimized medical image for diagnosis. In some examples, composite scores above a particular threshold value may be indicative of optimal images for diagnosis. In some examples, composite scores for each leg may be derived from separate digital medical images to assess whether hip dysplasia exists in either hip of a dog.

The method of flowchart 200 may further include generating and transmitting to a user computing device, computer-executable instructions configured to cause the user computing device to construct and display a user interface that presents the first score and/or at least a portion of the digital medical image. In some cases, the output first score may correspond to a particular suggested medical diagnosis (e.g., whether hip dysplasia is present). This may further be output to the user.

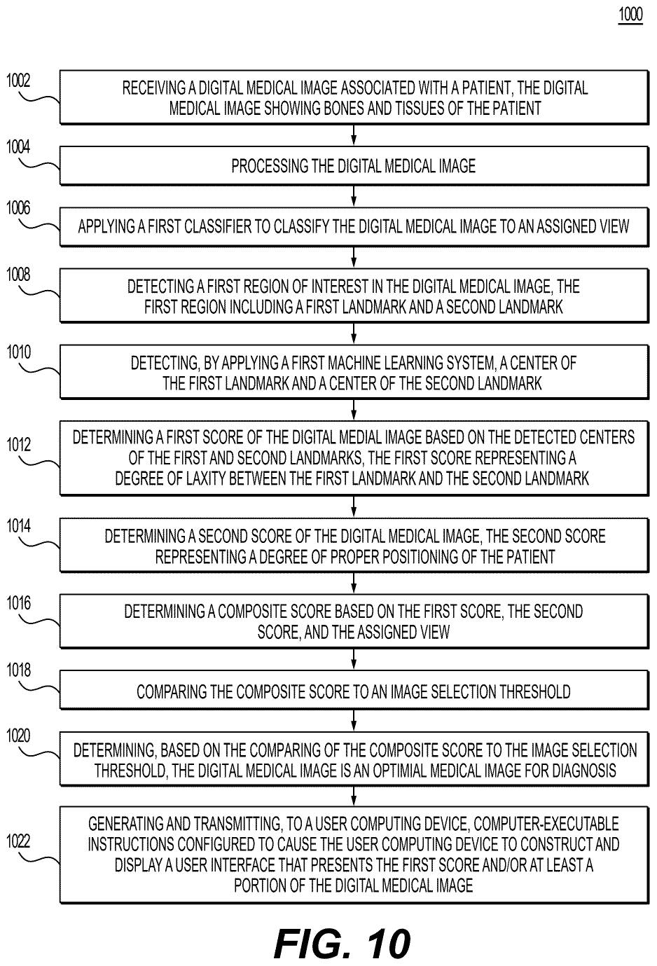

FIG. 10 depicts an exemplary flowchart 1000 of a process for processing a digital medical image to perform a diagnostic measurement, according to one or more embodiments. The process described in FIG. 10 may be implemented by the environment 100 of FIG. 1.

At step 1002, the dysplasia diagnosis management platform 120 may receive a digital medical image associated with a patient (or a subject), the digital medical image showing bones and tissues of the patient. The digital medical image may be a radiograph image. The digital medical image may further show medical equipment applied to the patient for diagnosis.

At step 1004, the dysplasia diagnosis management platform 120 may process the digital medical image. Processing the digital medical image may include determining a relevant region of the digital medical image, wherein the relevant region identifies a hip and a surrounding area of the hip of the patient. Processing the digital medical image may include determining, by applying a rotation classifier machine learning system, a rotation angle associated with the digital medical image, and rotating the digital medical image based on the rotation angle to properly orient the digital medical image. Processing the digital medical image may further include applying a classifier to the digital medical image to confirm the digital medical image is of a relevant view of the patient, wherein the relevant view includes a compression view, a distraction view, or a hip-extended view.

At step 1006, the classifier module 120D may apply a first classifier to classify the digital medical image to an assigned view. This may include classifying the digital medical image as one of a compression view, a distraction view, or a hip-extended view.

At step 1008, the dysplasia diagnosis management platform 120 may detect a first region of interest in the digital medical image, the first region including a first landmark and a second landmark. This may include determining an area of the digital medical image that includes a femoral head, an acetabulum, and a surrounding region of the femoral head and the acetabulum, wherein the first landmark is the femoral head (and its surrounding region) and the second landmark is the acetabulum (and its surrounding region). The detected first region of interest may be indicated using a bounding box on the digital medical image.

At step 1010, the DI calculation module 120E may detect, by applying a first machine learning system, a center of the first landmark and a center of the second landmark. The first machine learning system may be a convolutional neural network configured to perform image segmentation.

At step 1012, the DI calculation module 120E may determine a first score of the digital medical image based on the detected centers of the first and second landmarks, the first score representing a degree of laxity between the first landmark and the second landmark. Determining the first score may include determining a distance D representing a distance between the center of the first landmark and the center of the second landmark, determining a radius R representing a radius of the first landmark, and dividing the distance D by the radius R. The first score may be a DI.

At step 1014, the landmark detection module 120F may determine a second score of the digital medical image, the second score representing a degree of proper positioning of the patient. This may include determining a set of landmarks on the digital medical image, connecting the set of landmarks based on a predetermined sequence to form a set of edges, and determining the second score based on the set of edges. The second score may represent a degree of proper positioning of the patient's femoral head relative to the medical equipment in the digital medical image for diagnosis. The second score may be determined using a second machine learning system, the second machine learning system being a convolutional pose machine configured to analyze the degree of proper positioning of the patient relative to medical equipment applied to the patient for diagnosis.

The second score may include one or more of a landmark score representing a ratio of valid landmarks detected, a device score indicating presence of medical equipment in the digital medical image, an alignment score indicating a degree of alignment between one or more legs of the patient represented by a first portion of the set of the edges and medical equipment represented by a second portion of the set of the edges, or a femoral head score indicating whether the first landmark is positioned between a set of bars of the medical equipment. The device score, the alignment score, and the femoral head score may be calculated when the assigned view to which the digital medical image is classified is a distraction view image.

At step 1016, the scoring module 120G may determine a composite score based on one or more of the first score, the second score, and the assigned view.

At step 1018, the scoring module 120G may compare the composite score to an image selection threshold.

At step 1020, the scoring module 120G may determine, based on the comparing of the composite score to the image selection threshold, the digital medical image is an optimal medical image for diagnosis.

At step 1022, the server system 115 may generate and transmit to a user computing device, computer-executable instructions configured to cause the user computing device to construct and display a user interface that presents the first score and/or at least a portion of the digital medical image.

Although the method described above in reference FIG. 10 refers to “a” digital medical image, it should be noted that, as will be apparent to a person of ordinary skill in the art, each of the steps illustrated in FIG. 10 may be applied to (or performed for) a plurality of digital medical images. In this way, at step 1016, a plurality of composite scores may be determined for a plurality of corresponding digital medical images. At step 1018, the plurality of composite scores may be compared to the image selection threshold, and the images associated with composite scores equal to or greater than the image selection threshold may be determined optimal or well-suited for DI determination and/or diagnosis. The dysplasia diagnosis management platform 120 may select these images for viewing or consideration by the user (e.g., a veterinarian), and may output a list of the selected images, and/or the images themselves, to the user. In some embodiments, the dysplasia diagnosis management platform 120 may determine DI for each of the selected images automatically, and output the calculated Dis to the user in association with the corresponding images.

In general, any process discussed in this disclosure that is understood to be computer-implementable, such as the processes described in reference to FIG. 2, may be performed by one or more processors of a computer system, such as one or more components of the environment 100, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such one or more components of the environment 100, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

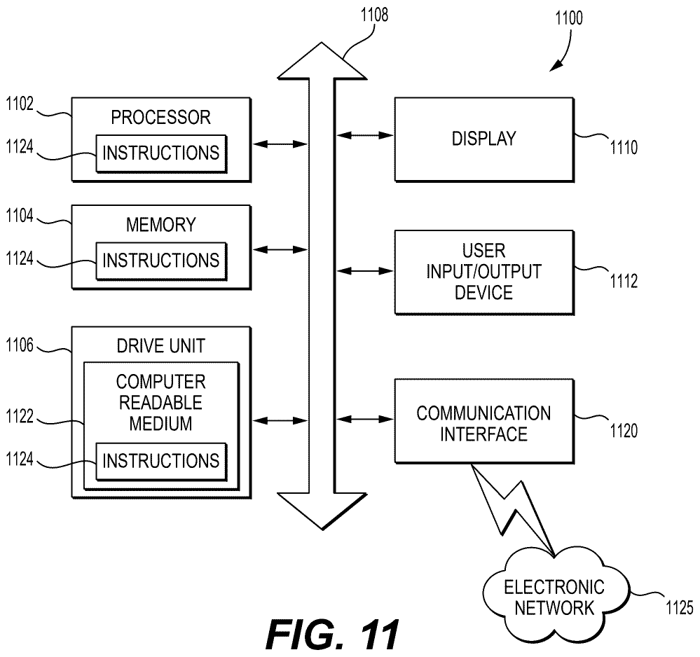

FIG. 11 is a simplified functional block diagram of a computer system 1100 that may be configured as a computing device for executing any processes or operations described herein, including the process illustrated in FIGS. 2 and 3, according to exemplary aspects of the present disclosure. In various aspects, any of the systems herein may be an assembly of hardware including, for example, a data communication interface 1120 for packet data communication. The platform also may include a central processing unit (“CPU”) 1102, in the form of one or more processors, for executing program instructions. The platform may include an internal communication bus 1108, and a storage unit 1106 (such as ROM, HDD, SDD, etc.) that may store data on a computer readable medium 1122, although the computer system 1100 may receive programming and data via network communications via electronic network 1125 (e.g., voice, video, audio, images, or any other data over the electronic network 1125). The computer system 1100 may also have a memory 1104 (such as RAM) storing instructions 1124 for executing techniques presented herein, although the instructions 1124 may be stored temporarily or permanently within other modules of computer system 1100 (e.g., processor 1102 and/or computer readable medium 1122). The computer system 1100 also may include input and output ports 1112 and/or a display 1110 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some aspects described herein include some but not other features included in other aspects, combinations of features of different aspects are meant to be within the scope of the invention, and form different aspects, as would be understood by those skilled in the art. For example, in the following claims, any of the claimed aspects can be used in any combination.

Thus, while certain aspects have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A method for processing a digital medical image to perform a diagnostic measurement, the method comprising:
receiving a digital medical image associated with a patient, the digital medical image showing bones and tissues of the patient;
processing the digital medical image;
applying a first classifier to classify the digital medical image to an assigned view;
detecting a first region of interest in the digital medical image, the first region including a first landmark and a second landmark;
detecting, by applying a first machine learning system, a center of the first landmark and a center of the second landmark;
determining a first score of the digital medical image based on the detected centers of the first and second landmarks, the first score representing a degree of laxity between the first landmark and the second landmark;
determining a second score of the digital medical image, the second score representing a degree of proper positioning of the patient, determining the second score including:
determining a set of landmarks on the digital medical image;
connecting the set of landmarks based on a predetermined sequence to form a set of edges; and
determining the second score based on the set of edges;
determining a composite score based on the first score and the second score;
comparing the composite score to an image selection threshold;
determining, based on the comparing of the composite score to the image selection threshold, the digital medical image is an optimal medical image for diagnosis; and
generating and transmitting, to a user computing device, computer-executable instructions configured to cause the user computing device to construct and display a user interface that presents the first score and/or at least a portion of the digital medical image.

2. The method of claim 1, wherein the digital medical image is a radiograph image.

3. The method of claim 1, wherein processing the digital medical image includes:
determining a relevant region of the digital medical image, wherein the relevant region identifies a hip and a surrounding area of the hip of the patient.

4. The method of claim 1, wherein processing the digital medical image includes:
determining, by applying a rotation classifier machine learning system, a rotation angle associated with the digital medical image; and
rotating the digital medical image based on the rotation angle to properly orient the digital medical image.

5. The method of claim 1, wherein processing the digital medical image includes:
applying the first classifier to the digital medical image to confirm the digital medical image is of a relevant view of the patient, wherein the relevant view includes a compression view, a distraction view, or a hip-extended view of a hip region of the patient.

6. The method of claim 1, wherein applying the first classifier to classify the digital medical image to an assigned view further includes:
classifying the digital medical image as one of a compression view, a distraction view, or a hip-extended view.

7. The method of claim 1, wherein detecting the first region of interest in the digital medical further includes:
determining an area of the digital medical image that includes a femoral head, an acetabulum, and a surrounding region of the femoral head and the acetabulum, wherein the first landmark is the femoral head and the second landmark is the acetabulum.

8. The method of claim 1, wherein the first machine learning system is a convolutional neural network configured to perform image segmentation.

9. The method of claim 1, wherein determining the first score includes:
determining a distance D representing a distance between the center of the first landmark and the center of the second landmark;
determining a radius R representing a radius of the first landmark; and
dividing the distance D by the radius R.

10. The method of claim 1, wherein the first score is a distraction index.

11. The method of claim 1, wherein the digital medical image further shows medical equipment applied to the patient for diagnosis, and wherein the second score represents a degree of proper positioning of a femoral head of the patient relative to the medical equipment in the digital medical image for diagnosis.

12. The method of claim 1, wherein the second score is determined using a second machine learning system, the second machine learning system being a convolutional pose machine configured to analyze the degree of proper positioning of the patient relative to medical equipment applied to the patient for diagnosis.

13. The method of claim 1, wherein the second score includes:
a landmark score representing a ratio of valid landmarks detected;
a device score indicating presence of medical equipment in the digital medical image;
an alignment score indicating a degree of alignment between one or more legs of the patient represented by a first portion of the set of the edges and medical equipment represented by a second portion of the set of the edges; and
a femoral head score indicating whether the first landmark is positioned between a set of bars of the medical equipment,
wherein the device score, the alignment score, and the femoral head score are calculated when the assigned view to which the digital medical image is classified as a distraction view image.

14. A computer system for processing a digital medical image to perform a diagnostic measurement, the computer system comprising:
at least one memory storing instructions; and
at least one processor configured to execute the instructions to perform operations comprising:
receiving a digital medical image associated with a patient, the digital medical image showing bones and tissues of the patient;
processing the digital medical image;
applying a first classifier to classify the digital medical image to an assigned view;
detecting a first region of interest in the digital medical image, the first region including a first landmark and a second landmark;
detecting, by applying a first machine learning system, a center of the first landmark and a center of the second landmark;
determining a first score of the digital medical image based on the detected centers of the first and second landmarks, the first score representing a degree of laxity between the first landmark and the second landmark;
determining a second score of the digital medical image, the second score representing a degree of proper positioning of the patient, determining the second score including:
determining a set of landmarks on the digital medical image;
connecting the set of landmarks based on a predetermined sequence to form a set of edges; and
determining the second score based on the set of edges;
determining a composite score based on the first score and the second score;
comparing the composite score to an image selection threshold;
determining, based on the comparing of the composite score to the image selection threshold, the digital medical image is an optimal medical image for diagnosis; and
generating and transmitting, to a user computing device, computer-executable instructions configured to cause the user computing device to construct and display a user interface that presents the first score and/or at least a portion of the digital medical image.

15. The system of claim 14, wherein the digital medical image is a radiograph image.

16. The system of claim 14, wherein processing the digital medical image includes:
determining a relevant region of the digital medical image, wherein the relevant region identifies a hip and a surrounding area of the hip of the patient.

17. The system of claim 14, wherein processing the digital medical image includes:
determining, by applying a rotation classifier machine learning system, a rotation angle associated with the digital medical image; and
rotating the digital medical image based on the rotation angle to properly orient the digital medical image.

18. The system of claim 14, wherein processing the digital medical image includes:
applying the first classifier to the digital medical image to confirm the digital medical image is of a relevant view of the patient, wherein the relevant view includes a compression view, a distraction view, or a hip-extended view of a hip region of the patient.

19. The system of claim 14, wherein applying the first classifier to classify the digital medical image to an assigned view further includes:
classifying the digital medical image as one of a compression view, a distraction view, or a hip-extended view.

20. A non-transitory computer-readable medium storing instructions that, when executed by a processor, cause the processor to perform operations for processing a digital medical image to perform a diagnostic measurement, the operations comprising:
receiving a digital medical image associated with a patient, the digital medical image showing bones and tissues of the patient;
processing the digital medical image;
applying a first classifier to classify the digital medical image to an assigned view;
detecting a first region of interest in the digital medical image, the first region including a first landmark and a second landmark;
detecting, by applying a first machine learning system, a center of the first landmark and a center of the second landmark;
determining a first score of the digital medical image based on the detected centers of the first and second landmarks, the first score representing a degree of laxity between the first landmark and the second landmark;
determining a second score of the digital medical image, the second score representing a degree of proper positioning of the patient, determining the second score including:
determining a set of landmarks on the digital medical image;
connecting the set of landmarks based on a predetermined sequence to form a set of edges; and
determining the second score based on the set of edges;
determining a composite score based on the first score and the second score;
comparing the composite score to an image selection threshold;
determining, based on the comparing of the composite score to the image selection threshold, the digital medical image is an optimal medical image for diagnosis; and
generating and transmitting, to a user computing device, computer-executable instructions configured to cause the user computing device to construct and display a user interface that presents the first score and/or at least a portion of the digital medical image.
